(19) United States
(12) Patent Application Publication
(10) Pub. No.: US 2014/0142360 A1
(43) Pub. Date: May 22, 2014

(54) BULK ETHYLENE OLIGOMERIZATION USING A LOW CONCENTRATION OF CHROMIUM CATALYST AND THREE-PART ACTIVATOR

(71) Applicants: Stephen John Brown, Calgary (CA); Charles Ashton Garret Carter, Calgary (CA); P. Scott Chisholm, Calgary (CA); Peter Zoricak, Calgary (CA); Oleksiy Golovchenko, Airdrie (CA)

(72) Inventors: Stephen John Brown, Calgary (CA); Charles Ashton Garret Carter, Calgary (CA); P. Scott Chisholm, Calgary (CA); Peter Zoricak, Calgary (CA); Oleksiy Golovchenko, Airdrie (CA)

(73) Assignee: NOVA CHEMICALS (INTERNATIONAL) S.A., Fribourg (CH)

(21) Appl. No.: 14/232,038

(22) PCT Filed: Jul. 25, 2012
(86) PCT No.: PCT/CA2012/000694

§ 371 Date: Jan. 10, 2014

(30) Foreign Application Priority Data
Jul. 26, 2011 (CA) 2747501

(51) Int. Cl. C07C 2/30

(52) U.S. Cl. CPC C07C 2/30
USPC 585/512

(57) ABSTRACT

This invention enables the “bulk” oligomerization of ethylene (i.e. the oligomerization of ethylene in the presence of the oligomer product) using a catalyst system comprising 1) a very low concentration of a chromium catalyst and 2) a three part activator. The chromium catalyst contains a diphosphine ligand, preferably a so called P—N—P ligand. The activator includes an aluminoxane, trimethyl aluminum, and triethyl aluminum.

### TECHNICAL FIELD

This invention relates to selective ethylene oligomerization reactions.

### BACKGROUND ART

Alpha olefins are commercially produced by the oligomerization of ethylene in the presence of a simple alkyl aluminum catalyst (in the so called “chain growth” process) or alternatively, in the presence of an organometallic nickel catalyst (in the so called Shell Higher Olefins, or “SHOP” process). Both of these processes typically produce a crude oligomer product having a broad distribution of alpha olefins with an even number of carbon atoms (i.e. butene-1, hexene-1, octene-1 etc.). The various alpha olefins in the crude oligomer product are then typically separated in a series of distillation columns. Butene-1 is generally the least valuable of these olefins as it is also produced in large quantities as a by-product in various cracking and refining processes. Hexene-1 and octene-1 often command comparatively high prices because these olefins are in high demand as comonomers for linear low density polyethylene (LLDPE).

Technology for the selective trimerization of ethylene to hexene-1 has been recently put into commercial use in response to the demand for hexene-1. The patent literature discloses catalysts which comprise a chromium source and a pyrrolide ligand as being useful for this process—see, for example, U.S. Pat. No. 5,198,563 (Reagen et al., assigned to Phillips Petroleum).

Another family of highly active trimerization catalysts is disclosed by Wass et al. in WO 02/04119 (now U.S. Pat. Nos. 7,143,633 and 6,800,702). The catalysts disclosed by Wass et al. are formed from a chromium source and a chelating diphosphine ligand and are described in further detail by Carter et al. (Chem. Comm. 2002, p 858-9). As described in the Chem. Comm. paper, these catalysts preferably comprise a diphosphine ligand in which both phosphine atoms are bonded to two phenyl groups that are each substituted with an ortho-methoxy group. Hexene-1 is produced with high activity and high selectivity by these catalysts.

Similar diphosphine/tetraphenyl ligands are disclosed by Blann et al. in WO04/056478 and WO 04/056479 (now US 2006/0229480 and US 2006/0173226). However, in comparison to the ligands of Wass et al., the disphosphine/tetraphenyl ligands disclosed by Blann et al. generally do not contain polar substituents in ortho positions. The “tetraphenyl” diphosphine ligands claimed in the '480 application must not have ortho substituents (of any kind) on all four of the phenyl groups and the “tetraphenyl” diphosphine ligands claimed in '226 are characterized by having a polar substituent in a meta or para position. Both of these approaches are shown to reduce the amount of hexenes produced and increase the amount of octene (in comparison to the ligands of Wass et al.). Other bridged diphosphine ligands that are useful for the selective oligomerization of ethylene are disclosed in the literature.

The above described chromium/diphosphine catalysts generally require an activator or catalyst in order to achieve meaningful rates of oligomerization. Aluminoxane are well known activators for this catalyst system. Methylaluminoxane (“MAO”)—which is made from trimethyl aluminum (TMA)—is generally preferred in terms of activity but suffers from a cost disadvantage. Accordingly, attempts have been made to reduce the cost of MAO activation by the additional use of less expensive aluminum alkyls such as triethyl aluminum (TEAL) or triisobutyl aluminum (TIBAL). Work in this area is disclosed in by Dixon et al. in WO2008/146215. The activation system of Dixon et al. requires a two stage activation procedure.

The use of additional process solvent has also been shown to increase reaction rates. In particular, WO 2005/123633 (Dixon et al.) illustrates that the use of cylcohexane or methylcyclohexane solvent can increase the rate of MAO cocatalyzed oligomerization reactions. This has the advantage of lowering catalyst costs but the disadvantage of requiring solvent separation from the oligomer product.

We have now discovered that exceptionally high catalyst activities can be obtained in the absence of additional cyclohexane when very low chromium concentrations are used in combination with a three part activator system that contains MAO, TMA and TEAL.

### DISCLOSURE OF INVENTION

In one embodiment, the present invention provides

### BEST MODE FOR CARRYING OUT THE INVENTION

### Part A Catalyst System

The preferred catalyst system used in the process of the present invention must contain three essential components, namely:

(i) a diphosphine ligand;

(ii) a source of chromium that coordinates to the ligand; and

(iii) a three part activator.

Preferred forms of each of these components are discussed below.

### (i) Ligand Used in the Oliciomerization Process

In general, the ligand used in the oligomerization process of this invention is defined by the formula (R¹)(R²)—P¹-bridge-P²(R³)(R⁴) wherein R¹, R², R³ and R⁴ are independently selected from the group consisting of hydrocarbyl and heterohydrocarbyl and the bridge is a divalent moiety that is bonded to both phosphorus atoms.

The term hydrocarbyl as used herein is intended to convey its conventional meaning—i.e. a moiety that contains only carbon and hydrogen atoms. The hydrocarbyl moiety may be a straight chain; it may be branched (and it will be recognized by those skilled in the art that branched groups are sometimes referred to as “substituted”); it may be saturated or contain unsaturation and it may be cyclic. Preferred hydrocarbyl groups contain from 1 to 20 carbon atoms. Aromatic groups—especially phenyl groups—are especially preferred. The phenyl may be unsubstituted (i.e. a simple C₆H₅ moiety) or contain substituents, particularly at an ortho (or “o”) position.

Similarly, the term heterohydrocarbyl as used herein is intended to convey its conventional meaning—more particularly, a moiety that contains carbon, hydrogen and at least one heteroatom (such as O, N, R and S). The heterohydrocarbyl groups may be straight chain, branched or cyclic structures. They may be saturated or contain unsaturation. Preferred heterohydrocarbyl groups contain a total of from 2 to 20 carbon+heteroatoms (for clarity, a hypothetical group that contains 2 carbon atoms and one nitrogen atom has a total of 3 carbon+heteroatoms).

It is preferred that each of R¹, R², R³ and R⁴ is a phenyl group (with an optional substituent in an ortho position on one or more of the phenyl groups). Highly preferred ligands are those in which R¹ to R⁴ are independently selected from the group consisting of phenyl, o-methylphenyl (i.e. ortho-methylphenyl), o-ethylphenyl, o-isopropylphenyl and o-fluorophenyl. It is especially preferred that none of R¹ to R⁴ contains a polar substituent in an ortho position. The resulting ligands are useful for the selective tetramerization of ethylene to octene-1 with some co product hexene also being produced. The term “bridge” as used herein with respect to the ligand refers to a divalent moiety that is bonded to both of the phosphorus atoms in the ligand—in other words, the “bridge” forms a link between P¹ and P². Suitable groups for the bridge include hydrocarbyl and an inorganic moiety selected from the group consisting of N(CH₃)—N(CH₃)—, —B(R⁶)—, —Si(R⁶)₂—, —P(R⁶)— or —N(R⁶)— where R⁶ is selected from the group consisting of hydrogen, hydrocarbyl and halogen.

It is especially preferred that the bridge is —N(R⁵)— wherein R⁵ is selected from the group consisting of hydrogen, alkyl, substituted alkyl, aryl, substituted aryl, aryloxy, substituted aryloxy, halogen, alkoxycarbonyl, carbonyloxy, alkoxy, aminocarbonyl, carbonylamino, dialkylamino, silyl groups or derivatives thereof and an aryl group substituted with any of these substituents. A highly preferred bridge is amino isopropyl (i.e. when R⁵ is isopropyl).

In one embodiment, two different types of ligands are used to alter the relative amounts of hexene and octene being produced. For clarity: the use of a ligand that produces predominantly hexene may be used in combination with a ligand that produces predominantly octene.

### (ii) Chromium Source

Any source of chromium that coordinates to the ligand and which allows the oligomerization process of the present invention to proceed may be used. Preferred chromium sources include chromium trichloride; chromium (III) 2-ethylhexanoate; chromium (III) acetylacetonate and chromium carbonyl complexes such as chromium hexacarbonyl. It is preferred to use very high purity chromium compounds as these should generally be expected to minimize undesirable side reactions. For example, chromium acetylacetonate having a purity of higher than 99% is commercially available (or may be readily produced from 97% purity material—using recrystallization techniques that are well known to those skilled in the art).

Catalyst systems comprising the above described liquids and a source of chromium are well known for the oligomerization of ethylene. The chromium concentrations that are typically disclosed in the relevant prior art are generally from 20 to 400 micromolar. The present invention requires a lower chromium concentration of from 0.5 to 8 micromolar, especially from 0.5 to 5 micromolar.

(iii) Three Part Activator

The three part activator of this invention includes

a) an aluminoxane; b) trimethyl aluminum and c) triethyl aluminum.

Aluminoxanes are well known, commercially available items of commerce. They may be prepared by the controlled addition of water to an alkyl aluminum compound such as TMA or TIBAL. Non-hydrolytic techniques to prepare aluminoxanes are also reported in the literature and are believed to be used by the AKZO Nobel Company to produce certain commercial products.

The use of methylaluminoxane (MAO) is preferred. It will be recognized by those skilled in the art that some commercially available MAO may be made using both of TMA and a higher alkyl aluminum (such as TIBAL) as starting materials in order to improve the solubility of the resulting MAO (in comparison to a MAO made solely from TMA). Those MAO's are generally referred to as “modified MAO's” and they are suitable for use in this invention.

It will also be recognized that commercially available MAO typically contains some “residual” or “free” TMA that is associated with the MAO. This TMA has been reported to influence the behavior of ethylene polymerization catalysts that are activated by MAO. Accordingly, it is known to treat MAO with a “modifier” that reacts with the free TMA in order to improve polymerization reactions (see for example, Collins et al.). We have conducted similar/analogous experiments with oligomerization catalysts and observed a profoundly negative effect—specifically, the oligomerization activity is reduced and/or the formation of by-product polymer is increased. We have not been able to mitigate these problems by the addition of a higher aluminum alkyl (such as TEAL). Accordingly, the use of TMA is necessary in this invention. The required amount of TMA is generally present in commercially available MAO, as described above. The use of additional TMA (i.e. further TMA, beyond that contained in the MAO) is also contemplated.

Both of the TMA and MAO are expensive materials. By comparison, the current commercial price of TEAL is less than half of TMA or MAO (on the basis of cost per unit weight of aluminum). It has previously been reported that the addition of TEAL to MAO (prior to contact with the oligomerization catalyst) can cause a large reduction in the activity of the catalyst (see WO 2008/146215). In contrast, the three part activator of the present invention (i.e. an aluminoxane, TMA and TEAL) may be pre-mixed, provided that 1) the chromium concentration is low (from 0.5 to 8 micromolar) and 2) the oligomerization is conducted in the presence of octene.

In general, the amount of TEAL is sufficient to provide from about 10 to 70% of the total aluminum that is added to the process on a molar basis—i.e.: (the moles of aluminum contained in TEAL)÷(the moles of aluminum contained in TEAL+TMA+MAO)×100% is from 10 to 70%.

More preferably, and stated in a different manner, the TEAL provides from about 50 to 300 moles of aluminum per 100 moles of aluminum provided by the TMA and MAO. For example, if the total amount of aluminum provided by a “commercial” MAO is 100 moles (including both of the aluminum contained in the aluminoxane and the “free TMA”), then it is preferred to add additional TEAL in an amount from 50 to 300 moles of aluminum.

The amount of aluminoxane, TMA and TEAL is preferably sufficient to provide a total Al:Cr molar ratio of from 200:1 to 1500:1, especially from 300:1 to 1000:1, for batch reactions and up to 2500:1 for continuous reactions. The use of Al:Cr as high as 2500:1 is also within the scope of the invention, especially when very low Cr concentrations are used. It is also preferred that the aluminum concentration in the reactor is at least 2 millimolar (2000 micromolar) because lower levels of aluminum may not be sufficient to “scavenge” impurities.

### Part B Process Conditions

The chromium and ligand may be present in any molar ratio which produces oligomer, preferably between 100:1 and 1:100, and most preferably from 10:1 to 1:10, particularly 3:1 to 1:3. Generally the amounts of (i) and (ii) are approximately equal, i.e. a ratio of between 1.5:1 and 1:1.5.

Suitable solvents for contacting the components of the catalyst or catalyst system include, but are not limited to, hydrocarbon solvents such as heptane, toluene, 1-hexene and the like, and polar solvents such as diethyl ether, tetrahydrofuran, acetonitrile, dichloromethane, chloroform, chlorobenzene, acetone and the like.

The catalyst components may be mixed together in the oligomerization reactor, or—alternatively—some or all of the catalyst components may be mixed together outside of the oligomerization reactor. Suitable method of catalyst synthesis are illustrated in the examples. Some catalyst components have comparatively low solubility in octene. For example, MAO that is made solely with trimethylaluminum (as opposed to “modified MAO” which also contains some higher alkyl aluminum, such as triisobutyl aluminum) is less soluble in octene than in some cyclic hydrocarbons such as xylene or tetralin. Accordingly, when one or more catalyst components are mixed together outside of the oligomerization reactor, the use of xylene or tetralin as the solvent may be preferred. The xylene may be a mixture of ortho, meta and para isomers—i.e. it is not necessary to use a pure isomer.

A variety of methods are known to purify solvents used in the oligomerization process including use of molecular sieves (3A), adsorbent alumina and supported de-oxo copper catalyst. Several configurations for the purifier system are known and depend on the nature of the impurities to be removed, the purification efficiency required and the compatibility of the purifier material and the process solvent. In some configurations, the process solvent is first contacted with molecular sieves, followed by adsorbent alumina, then followed by supported de-oxo copper catalyst and finally followed by molecular sieves. In other configurations, the solvent is first contacted with molecular sieves, followed by adsorbent alumina and finally followed by molecular sieves. In yet another configuration, the solvent is contacted with adsorbent alumina.

In a preferred process, the amount of solvent that is added is very low (and is provided in an amount that is required to comfortably add the catalyst and activator to the process). This type of process is generally referred to as a “bulk process”, in the sense that the process is conducted using the oligomerization product as the reaction medium. Suitable temperatures range from 10° C. to +300° C. preferably from 10° C. to 100° C., especially from 20 to 80° C. Suitable pressures are from atmospheric to 800 atmospheres (gauge) preferably from 5 atmospheres to 100 atmospheres, especially from 10 to 50 atmospheres for batch processes and up to 90-100 atmospheres for continuous process.

Irrespective of the process conditions employed, the oligomerization is typically carried out under conditions that substantially exclude oxygen, water, and other materials that act as catalyst poisons. In addition, the reactor is preferably purged with a nonreactive gas (such as nitrogen or argon) prior to the introduction of catalyst. A purge with a solution of MAO and/or aluminum alkyl may also be employed to lower the initial level of catalyst poisons. Also, oligomerizations can be carried out in the presence of additives to control selectivity, enhance activity and reduce the amount of polymer formed in oligomerization processes. Potentially suitable additives include, but are not limited to, hydrogen or a halide source (especially the halide sources disclosed in U.S. Pat. No. 7,786,336, Zhang et al.). Other (optional) additives include antistatic agents (such as the polysulfone polymer sold under the trademark Stadis®) and/or fluorocarbons to mitigate reaction fouling; or amines to alter the hexene/octene ratio of the product oligomer (as disclosed in U.S. application 20090118117, Elowe et al.). The use of hydrogen is especially preferred because it has been observed to reduce the amount of polymer that is formed. The preferred catalysts of this invention predominantly produce octene with some hexane (as shown in the examples) but smaller quantities of butene and C₁₀+ olefins are also produced. The crude product stream may be separated into various fractions using, for example, a conventional distillation system. It is within the scope of this invention to recycle the “whole” oligomer product or some fraction(s) thereof to the reaction for use as an oligomerization diluent. For example, by recycling a butene rich stream it might be possible to lower the refrigeration load in distillation. Alternatively, the C₁₀+ fraction might be preferentially recycled to improve the solubility of one or more components of the catalyst system.

Techniques for varying the distribution of products from the oligomerization reactions include controlling process conditions (e.g. concentration of components (i)-(iii), reaction temperature, pressure, residence time) and properly selecting the design of the process and are well known to those skilled in the art.

In another embodiment, a catalyst that produces ethylene homopolymer is deliberately added to the reactor in an amount sufficient to convert from 1 to 5 weight % of the ethylene feed to an ethylene homopolymer. This catalyst is preferably supported. The purpose is to facilitate the removal of by-product polyethylene.

The ethylene feedstock for the oligomerization may be substantially pure or may contain other olefinic impurities and/or ethane. One embodiment of the process of the invention comprises the oligomerization of ethylene-containing waste streams from other chemical processes or a crude ethylene/ethane mixture from a cracker as more fully described in co-pending Canadian patent application 2,708,011 (Krzywicki et al.).

The feedstock is preferably treated to remove catalyst poisons (such as oxygen, water and polar species) using techniques that are well known to those skilled in the art. The technology used to treat feedstocks for polymerizations is suitable for use in the present invention and includes the molecular sieves, alumina and de-oxo catalysts described above for analogous treatment of the process solvent.

### Reactor Systems

A general review of suitable reactors for selective oligomerization is provided first, followed by a detailed description of preferred reactor designs. There exist a number of options for the oligomerization reactor including batch, semi-batch, and continuous operation. Oligomerization reactions can generally be performed under a range of process conditions that are readily apparent to those skilled in the art. Evaporative cooling from one or more monomers or inert volatile liquids is but one (prior art) method that can be employed to effect the removal of heat from the reaction. The reactions may be performed in the known types of reactors, such as a plug-flow reactor, or a continuously stirred tank reactor (CSTR), or a loop reactor, or combinations thereof. A wide range of methods for effecting product, reactant, and catalyst separation and/or purification are known to those skilled in the art and may be employed: distillation, filtration, liquid-liquid separation, slurry settling, extraction, etc. One or more of these methods may be performed separately from the oligomerization reaction or it may be advantageous to integrate at least some with the reaction; a non-limiting example of this would be a process employing catalytic (or reactive) distillation. Also advantageous may be a process which includes more than one reactor, a catalyst kill system between reactors or after the final reactor, or an integrated reactor/separator/purifier. While all catalyst components, reactants, inerts, and products could be employed in the present invention on a once-through basis, it is often economically advantageous to recycle one or more of these materials; in the case of the catalyst system, this might require reconstituting one or more of the catalysts components to achieve the active catalyst system.

More specific reactor designs have been described in the patent literature:
a liquid phase reactor with “bubbling” ethylene feed is taught as a means to mitigate PE formation (WO 2009/060342, Kleingeld et al.);
a liquid phase reactor with an inert, condensable liquid is claimed as a means to improve temperature control (WO 2009/060343, Crildenhuys). The condensable liquid boils from the reaction liquid and is condensed overhead; and
the use of a liquid/gas phase reactor in which cooling coils are present in the gas phase head space is described in WO 2007/016996, Fritz et al.).

The present invention provides additional reactor designs for selective oligomerizations. The present invention is characterized (in part) by the requirement that a non adiabatic reactor system is used. The term “non adiabatic” means that heat is added to and/or removed from the oligomerization reactor. The term “reactor system” means that one or more reactors are employed (and the term “non adiabatic reactor system” means that at least one of the reactors is equipped with a heat exchanger that allows heat to be added to or removed from it). One embodiment relates to a CSTR with an external heat exchanger. A second embodiment relates to a tubular plug flow equipped with multiple feed ports for ethylene along the length of the reactor. A third embodiment relates to a combination of a CSTR followed by a tubular reactor. A fourth embodiment provides a loop reactor. A fifth embodiment provides a reactor having an internal cooling system (such as a draft tube reactor).

One preferred CSTR for use in the present invention is equipped at least one external heat exchanger—meaning that the heat exchanger surface(s) are not included within the walls of the CSTR. The term “heat exchanger” is meant to include its broad, conventional meaning. Most importantly, the heat exchanger will preferably be designed so as to allow heating of the reactor contents (which may be desirable during start up) and to provide heat removal during the oligomerization. A preferred external heat exchanger for a CSTR comprises a conventional shell and tube exchanger with a “process” side tube system and a shell for the exchange side. In one embodiment the “process side” (i.e. the side of the exchanger that contains the fluid from the oligomerization process) is a tube that exits the reactor and flows through the shell for heat exchange, then reenters the reactor with cooled (or heated) process fluid. For clarity: during an oligomerization reaction a portion of the hot reactor contents or “process fluid” will flow from the reactor to the external heat exchanger, through a tube. The exterior of the tube comes into contact with cold fluid on the shell side of the exchanger, thus cooling the process fluid. The cooled process fluid is then returned to the reactor.

The use of two of more CSTR reactors in series is also contemplated. In particular, the use of a first CSTR having a small volume followed by a larger CSTR might be used to facilitate startup.

In another embodiment, a heat exchanger is located between two CSTRs. In this embodiment, the product from the first oligomerization reactor leaves that reactor through an exit tube. The oligomerization products in this exit tube are then directed through a heat exchanger. After being cooled by the heat exchanger, the oligomerization products are then directed into a second CSTR. Additional ethylene (and, optionally, catalyst) is added to the second CSTR and further oligomerization takes place.

The amount of heat generated by the oligomerization reaction is generally proportional to the amount of ethylene being oligomerized. Thus, at high rates of oligomerization, a high rate of coolant flow is required in the shell side of the exchanger.

In a highly preferred embodiment, the ethylene/solvent is fed to the CSTR through a plurality of feed ports. In one such embodiment, the feed is provided by way of a tubular ring that contains a plurality of holes and follows a circle around an interior diameter of the CSTR. The ethylene (and optional solvent or diluent) is preferably directed into liquid contained in the reactor (as opposed to gas) and even more preferably, the CSTR is operated in a liquid full mode. As used herein, the term “liquid full” means that the reactor is at least 90% full of liquid (by volume). More preferably, the ethylene is co-fed with hydrogen (i.e. hydrogen is added through the same feed part as the ethylene). Even more preferably, the CSTR is equipped with at least two impellers that are separated from each other along the length of the agitator shaft and the ethylene/hydrogen feed is directed to the tip of one impeller and the catalyst feed is directed to the tip of the second impeller that is located at a different point along the length of the agitator shaft.

Conventional baffles that run vertically along the interior wall of the CSTR may be included to enhance mixing.

The average feed velocity for the ethylene/solvent is preferably from 0.1 to 100 mm/s. Feed velocity is calculated by dividing the volumetric flow rate (mm³/s) by the total area of openings in the feed ports (mm²). High feed velocity (and a plurity of feed ports) helps to rapidly disperse the ethylene. Optimum feed velocity will, in general, be influenced by a number of variables—including reactor geometry, reactor agitation and production rates. The optimization of feed rates may require that the size and number of feed ports is changed—but such optimization and changes are well within the scope of those of ordinary skill in the art.

The CSTR is preferably operated in continuous flow mode—i.e. feed is continuously provided to the CSTR and product is continuously withdrawn.

The CSTR described above may be used to provide the high degree of temperature control that we have observed to be associated with a low degree of polymer formation.

In another embodiment, the CSTR is equipped with one or more of the mixing elements described in U.S. Pat. No. 6,319,996 (Burke et al.). In particular, Burke et al. disclose the use of a tube which has a diameter that is approximately equal to the diameter of the agitator of the CSTR. This tube extends along the length of the agitator shaft, thereby forming a mixing element that is often referred to as a “draft tube” by those skilled in the art. The reactor used in this invention may also employ the mixing helix disclosed by Burke et al. (which helix is located within the draft tube and forms a type of auger or Archimedes screw within the draft tube). The use of stationary, internal elements (to divide the CSTR into one or more zones) may also be employed. In one such example, two impellers are vertically displaced along the length of the agitation shaft i.e. one in the top part of the reactor and another in the bottom. An internal “ring” or “doughnut” is used to divide the CSTR into a top reaction zone and a bottom reaction zone. The ring is attached to the diameter of the CSTR and extends inwardly towards the agitation shaft to provide a barrier between the top and bottom reaction zones. A hole in the center of the ring allows the agitation shaft to rotate freely and provides a pathway for fluid flow between the two reactions zones. The use of such rings or doughnuts to divide a CSTR into different zones is well known to those skilled in the art of reactor design.

In another embodiment, two or more separate agitators with separate shafts and separate drives may be employed. For example, a small impeller might be operated at high velocity/high shear rate to disperse the catalyst and/or ethylene as it enters the reactor and a separate (larger) impeller with a draft tube could be used to provide circulation within the reactor.

An alternative reactor design is a tubular/plug flow reactor with an external heat exchanger. Tubular/plug flow reactors are well known to those skilled in the art. In general, such reactors comprise one or more tubes with a length/diameter ratio of from 10/1 to 1000/1. Such reactors are not equipped with active/powered agitators but may include a static mixer. Examples of static mixers include those manufactured and sold by Koch-Glitsch Inc. and Sulzer-Chemtech.

Tubular reactors for use in the present invention are preferably characterized by two features:
1) external cooling; and
2) the use of at least one incremental ethylene feed port along the length of the tubular reactor (i.e. in addition to the initial ethylene feed at the start of the tubular reactor).

In one embodiment, the tubular reactor is a so called “heat-exchange reactor” which is generally configured as a tube and shell heat exchanger. The oligomerization reaction occurs inside the tube(s) of this reactor. The shell side provides a heat exchange fluid (for the purposes described above, namely to heat the reaction during start up and/or to cool the reaction during steady state operations).

In one embodiment, the tubes are bent so as to form a type of static mixer for the fluid passing through the shell side. This type of heat exchanger is known to those skilled in the art and is available (for example) from Sulzer-Chemtech under the trade name SMR.

It is especially preferred that the Reynolds number of the reaction fluid that flows through the tube (or tubes) of the tubular reactor is from 2000 to 10,000,000. Reynolds number is a dimensionless number that is readily calculated using the following formula:

$$Re = \frac{pVL}{μ}$$

where:
V is the mean fluid velocity (SI units: m/s);
L is a characteristic linear dimension (e.g. internal diameter of tube);
μ is the dynamic viscosity of the fluid (Pa·s or N·s/m² or kg/(m·s)); and
p is the density of the fluid (kg/m³).

In one such embodiment a plurality of heat exchange reactors are connected in series. Thus, the process flow that exits the first reactor enters the second reactor. Additional ethylene is added to the process flow from the first reactor but additional catalyst is preferably not added.

In another embodiment, a CSTR is connected in series to a tubular reactor. One sub embodiment of this dual reactor system comprises a CSTR operated in adiabatic mode, followed by a tubular reactor having an external heat exchanger—in this embodiment the amount of ethylene that is consumed (i.e. converted to oligomer) in the CSTR is less than 50 weight % of the total ethylene that is consumed in the reactors. In another sub embodiment of this dual reactor system, a CSTR that is equipped with an external heat exchanger is connected to a downstream tubular reactor that is operated in adiabatic mode. In this embodiment, the amount of ethylene that is converted/consumed in the CSTR is in excess of 80 weight % of the ethylene that is consumed in the reactor. The tubular reactor may also have several different ports which allow the addition of catalyst killer/deactivator along the length of the reactor. In this manner, some flexibility is provided to allow the reaction to be terminated before the product exits from the reactor.

Another reactor design for use in the present invention is a loop reactor. Loop reactors are well known and are widely described in the literature. One such design is disclosed in U.S. Pat. No. 4,121,029 (Irvin et al.). The loop reactor disclosed by Irvin et al. contains a “wash column” that is connected to the upper leg of the loop reactor and is used for the collection of polymer. A similar “wash column” is contemplated for use in the present invention to collect by-product polymer (and/or supported catalyst). A hydrocyclone at the top end of the wash column may be used to facilitate polymer separation.

A fifth reactor design for use in the present invention is another type of heat exchange reactor in which the process side (i.e. where the oligomerization occurs) is the “shell side” of the exchanger. One embodiment of this reactor design is a so called “draft tube” reactor of the type reported to be suitable for the polymerization of butyl rubber. This type of reactor is characterized by having an impeller located near the bottom of the reactor, with little or no agitator shaft extending into the reactor. The impeller is encircled with a type of “draft tube” that extends upwards through the center of the reactor. The draft tube is open at the bottom (to allow the reactor contents to be drained into the tube, for upward flow) and at the top—where the reactor contents are discharged from the tube. A heat exchanger tube bundle is contained within the reactor and is arranged such that the tubes run parallel to the draft tube and are generally arranged in a concentric pattern around the draft tube. Coolant flows through the tubes to remove the heat of the reaction.

Monomer is preferably added by one or more feed ports that are located on the perimeter of the reactor (especially near the bottom of the reactor) and oligomerization product is withdrawn through at least one product exit port (preferably located near the top of the reactor). Catalyst is preferably added through a separate feed line that is not located close to any of the monomer feed ports(s) or product exit port(s). Draft tube reactors are well known and are described in more detail in U.S. Pat. No. 4,007,016 (Weber) and U.S. Pat. No. 2,474,592 (Palmer) and the references therein. FIG. 2 of U.S. Pat. No. 2,474,592 illustrates the use of a fluid flushing system to flush the agitator shaft in the vicinity of the agitator shaft seal. More specifically, a fluid chamber through the agitator shaft seal is connected to a source of flushing fluid (located outside of the reactor) and the channel terminates in the area where the agitator shaft enters the reactor. “Flushing fluid” is pumped through the channel to flush the base of the agitator and thereby reduce the amount of polymer build up at this location.

Another form of this type of reactor (i.e. in which the process is undertaken on the “shell” side of an internally heat exchanged reactor) is sold by ABB Lummus under the trademark Helixchanger®

Another known technique to reduce the level of fouling in a chemical reactor is to coat the reactor walls and/or internals and/or agitators with a low fouling material such as glass or polytetrafluoroethylene (PTFE). The use of coatings can be especially beneficial on high fouling areas such as agitator shafts and impellers.

### Reactor Control

The control systems required for the operation of CSTR's and tubular reactors are well known to those skilled in the art and do not represent a novel feature of the present invention. In general, temperature, pressure and flow rate readings will provide the basis for most conventional control operations. The increase in process temperature (together with reactor flow rates and the known enthalpy of reaction) may be used to monitor ethylene conversion rates. The amount of catalyst may be increased to increase the ethylene conversion (or decreased to decrease ethylene conversion) within desired ranges. Thus, basic process control may be derived from simple measurements of temperature, pressure and flow rates using conventional thermocouples, pressure meters and flow meters. Advanced process control (for example, for the purpose of monitoring product selectivity or for the purpose of monitoring process fouling factors) may be undertaken by monitoring additional process parameters with more advanced instrumentation. Known/existing instrumentation that may be employed include in-line/on-line instruments such as NIR infrared, Fourier Transform Infrared (FTIR), Raman, mid-infrared, ultra violet (UV) spectrometry, gas chromatography (GC) analyzer, refractive index, on-line densitometer or viscometer. The use of NIR or GC to measure the composition of the oligomerization reactor and final product composition is especially preferred.

The measurement may be used to monitor and control the reaction to achieve the targeted stream properties including but not limited to concentration, viscosity, temperature, pressure, flows, flow ratios, density, chemical composition, phase and phase transition, degree of reaction, polymer content, selectivity.

The control method may include the use of the measurement to calculate a new control set point. The control of the process will include the use of any process control algorithms, which include, but are not limited to the use of PID, neural networks, feedback loop control, forward loop control and adaptive control.

### Catalyst Deactivation, Catalyst Removal and Polymer Removal

In general, the oligomerization catalyst is preferably deactivated immediately downstream of the reactor as the product exits the reaction vessel. This is to prevent polymer formation and potential build up downstream of the reactor and to prevent isomerisation of the 1-olefin product to the undesired internal olefins. It is generally preferred to flash and recover unreacted ethylene before deactivation. However, the option of deactivating the reactor contents prior to flashing and recovering ethylene is also acceptable. The flashing of ethylene is endothermic and may be used as a cooling source. In one embodiment, the cooling provided by ethylene flashing is used to chill a feedstream to the reactor.

In general, many polar compounds (such as water, alcohols and carboxylic acids) will deactivate the catalyst. The use of alcohols and/or carboxylic acids is preferred—and combinations of both are contemplated. It is generally found that the quantity employed to deactivate the catalyst is sufficient to provide deactivator to metal (from activator) mole ratio between about 0.1 to about 4. The deactivator may be added to the oligomerization product stream before or after the volatile unreacted reagents/diluents and product components are separated. In the event of a runaway reaction (e.g. rapid temperature rise) the deactivator can be immediately fed to the oligomerization reactor to terminate the reaction. The deactivation system may also include a basic compound (such as sodium hydroxide) to minimize isomerization of the products (as activator conditions may facilitate the isomerization of desirable alpha olefins to undesired internal olefins).

Polymer removal (and, optionally, catalyst removal) preferably follows catalyst deactivation. Two “types” of polymer may exist, namely polymer that is dissolved in the process solvent and non-dissolved polymer that is present as a solid or “slurry”.

Solid/non-dissolved polymer may be separated using one or more of the following types of equipment: centrifuge; cyclone (or hydrocyclone), a decanter equipped with a skimmer or a filter. Preferred equipment include so called “self cleaning filters” sold under the name V-auto strainers, self cleaning screens such as those sold by Johnson Screens Inc. of New Brighton, Minn. and centrifuges such as those sold by Alfa Laval Inc. of Richmond, Va. (including those sold under the trade name Sharples).

Soluble polymer may be separated from the final product by two distinct operations. Firstly, low molecular weight polymer that remains soluble in the heaviest product fraction (C₂₀₊) may be left in that fraction. This fraction will be recovered as “bottoms” from the distillation operations (described below). This solution may be used as a fuel for a power generation system.

An alternative polymer separation comprises polymer precipitation caused by the removal of the solvent from the solution, followed by recovery of the precipitated polymer using a conventional extruder. The technology required for such separation/recovery is well known to those skilled in the art of solution polymerization and is widely disclosed in the literature.

In another embodiment, the residual catalyst is treated with an additive that causes some or all of the catalyst to precipitate. The precipitated catalyst is preferably removed from the product at the same time as by-product polymer is removed (and using the same equipment). Many of the catalyst deactivators listed above will also cause catalyst precipitation. In a preferred embodiment, a solid sorbent (such as clay, silica or alumina) is added to the deactivation operation to facilitate removal of the deactivated catalyst by filtration or centrifugation.

Reactor fouling (caused by deposition of polymer and/or catalyst residue) can, if severe enough, cause the process to be shut down for cleaning. The deposits may be removed by known means, especially the use of high pressure water jets or the use of a hot solvent flush. The use of an aromatic solvent (such as toluene or xylene) for solvent flushing is generally preferred because they are good solvents for polyethylene. The use of the heat exchanger that provides heat to the present process may also be used during cleaning operations to heat the cleaning solvent.

### Distillation

In one embodiment of the present invention, the oligomerization product produced from this invention is added to a product stream from another alpha olefins manufacturing process for separation into different alpha olefins. As previously discussed, “conventional alpha olefin plants” (wherein the term includes i) those processes which produce alpha olefins by a chain growth process using an aluminum alkyl catalyst, ii) the aforementioned “SHOP” process and iii) the production of olefins from synthesis gas using the so called Lurgi process) have a series of distillation columns to separate the “crude alpha product” (i.e. a mixture of alpha olefins) into alpha olefins (such as butene-1, hexene-1 and octene-1). The mixed hexene-octene product which is preferably produced in accordance with the present invention is highly suitable for addition/mixing with a crude alpha olefin product from an existing alpha olefin plant (or a “cut” or fraction of the product from such a plant) because the mixed hexene-octene product produced in accordance with the present invention can have very low levels of internal olefins. Thus, the hexene-octene product of the present invention can be readily separated in the existing distillation columns of alpha olefin plants (without causing the large burden on the operation of these distillation columns which would otherwise exist if the present hexene-octene product stream contained large quantities of internal olefins). As used herein, the term “liquid product” is meant to refer to the oligomers produced by the process of the present invention which have from 4 to (about) 20 carbon atoms.

In another embodiment, the distillation operation for the oligomerization product is integrated with the distillation system of a solution polymerization plant (as disclosed in Canadian patent application no. 2,708,011, Krzywicki et al.).

If toluene is present in the process fluid (for example, as a solvent for a MAO activator), it is preferable to add water to the “liquid product” prior to distillation to form a water/toluene azeotrope with a boiling point between that of hexene and octene.

The liquid product from the oligomerization process of the present invention preferably consists of from 20 to 80 weight % octenes (especially from 35 to 75 weight %) octenes and from 15 to 50 weight % (especially from 20 to 40 weight %) hexenes (where all of the weight % are calculated on the basis of the liquid product by 100%.

The preferred oligomerization process of this invention is also characterized by producing very low levels of internal olefins (i.e. low levels of hexene-2, hexene-3, octene-2, octene-3 etc.), with preferred levels of less than 10 weight % (especially less than 5 weight %) of the hexenes and octenes being internal olefins.

### Examples

The following abbreviations are used in the examples:

Å=Angstrom units
NMR=nuclear magnetic resonance
Et=ethyl
Bu=butyl
iPr=isopropyl
c*=comparative
rpm=revolutions per minute
GC=gas chromatography
Rₓ=reaction
Wt=weight
C₄'s=butenes
C₆'s=hexenes
C₈'s=octenes
PE=polyethylene

### Part I: Preferred Ligand Synthesis

### General

This section illustrates the synthesis of a preferred but non-limiting ligand for use in the present invention.

All reactions involving air and or moisture sensitive compounds were conducted under nitrogen using standard Schlenk or cannula techniques, or in a glovebox. Reaction solvents were purified prior to use (e.g. by distillation) and stored over activated 4 Å sieves. Diethylamine, triethylamine and isopropylamine were purchased from Aldrich and dried over 4 Å molecular sieves prior to use. 1-Bromo-2-fluorobenzene, phosphorus trichloride (PCl₃), hydrogen chloride gas and n-butyllithium were purchased from Aldrich and used as is. The methylalumoxane (MAO), 10 wt % Al in toluene, was purchased from Akzo and used as is. Deuterated solvents were purchased (toluene-d₈, THF-d₈) and were stored over 4 Å sieves. NMR spectra were recorded on a Bruker 300 MHz spectrometer (300.1 MHz for ¹H, 121.5 MHz for ³¹P, 282.4 for ¹⁹F).

### Preparation of Et₂NPCl₂

Et₂NH (50.00 mmol, 5.17 mL) was added dropwise to a solution of PCl₃ (25.00 mmol, 2.18 mL) in diethyl ether (will use “ether” from here) (200 mL) at −78° C. After the addition, the cold bath was removed and the slurry was allowed to warm to room temperature over 2 hours. The slurry was filtered and the filtrate was pumped to dryness. The residue was distilled (500 microns, 55° C.) to give the product in quantitative yield.

¹H NMR (δ, toluene-d₈): 2.66 (doublet of a quartets, 4H, J_{PH}=13 Hz, J_{HH}=7 Hz), 0.75 (triplet, 6H, J=7 Hz).

### Preparation of (ortho-F—C₆H₄)₂P-NEt₂

To solution of n-BuLi (17.00 mL of 1.6 M n-BuLi hexane solution, 27.18 mmol) in ether (100 mL) maintained at −85° C., was added dropwise a solution of 1-bromo-2-fluorobenzene (4.76 g, 27.18 mmol) in ether (40 mL) over 2 hours. After addition, the reaction flask was stirred for 1 hour at −78° C., resulting in a white slurry. Et₂NPCl₂ (2.36 g, 13.58 mmol) in ether (20 mL) was then added very slowly while the reaction temperature was maintained at −85° C. The reaction was allowed to warm to −10° C. overnight. Toluene (10 mL) was then added to the reaction flask and the volatiles were removed in vacuo. The residue was extracted with toluene and the solution was pumped to dryness. The crude product was distilled (300 microns, 100° C.) yielding 3.78 g (95%) of product. ¹H NMR (δ, THF-d₈): 7.40-7.01 (4 equal intense multiplets, 8H), 3.11 (doublets of quartet, 4H, J_{PH}=13 Hz, J_{HH}=7 Hz), 0.97 (triplet, 6H, J=7 Hz). ¹⁹F NMR (δ, THF-d₈): −163.21 (doublet of multiplets, J=48 Hz). GC-MS. M⁺=293.

### Preparation of (ortho-F—C₆H₄)₂PCI

Anhydrous HCl_{(g)} was introduced to the head space of an ethereal solution (100 mL) of (ortho-F—C₆H₄)P-NEt₂ (3.73 g, 12.70 mmol) to a pressure of 3 psi. A white precipitate formed immediately. The reaction was stirred for an additional 0.5 hours at which point the slurry was pumped to dryness to remove volatiles. The residue was re-slurried in ether (100 mL) and filtered. The filtrate was pumped to dryness yielding (ortho-F—C₆H₄)₂PCI as a colorless oil in quantitative yield. ¹H NMR (δ, THF-d₈): 7.60 (m, 4H), 7.20 (m, 2H), 7.08 (m, 2H). ¹⁹F NMR (δ, THF-d₈): −106.94 (doublet of multiplets, J=67 Hz).

### Preparation of (ortho-F—C₆H₄)₂PNH(i-Pr)

To a solution of (ortho-F—C₆H₄)PCI (1.00 g, 3.90 mmol) in ether (50 mL) and NEt₃ (3 mL) was added an ethereal solution of i-PrNH₂ (0.42 mL, 4.90 mmol) at −5° C. Immediate precipitate was observed. The slurry was stirred for 3 hours and filtered. The filtrate was pumped to dryness to give a colorless oil of (ortho-F—C₆H₄)PNH(i-Pr) in quantitative yield.

¹H NMR (δ, THF-d₈): 7.42 (m, 2H), 7.30 (m, 2H), 7.11 (m, 2H), 6.96 (m, 2H), 3.30 (septet, 1H, J=7 Hz), 2.86 (br s, 1H), 1.15 (d, 6H, J=7 Hz). ¹⁹F NMR (δ, THF-d₈): −109.85 (doublet of multiplets, J=40 Hz). GC-MS, M⁺=279.

### Preparation of (ortho-F—C₆H₄)₂PN(i-Pr)P(ortho-F—C₈H₄)₂ (“Ligand 1”)

To a solution of (ortho-F—C₆H₄)₂PNH(i-Pr) (3.90 mmol) [made from i-PrNH₂ and (ortho-F—C₆H₄)₂PCI (1.00 g, 3.90 mmol)] in ether (100 mL) maintained at −70° C. was added dropwise a solution of n-BuLi (2.43 mL of 1.6 M n-BuLi hexane solution, 3.90 mmol)). The mixture was stirred at −70° C. for 1 hour and allowed to warm to −10° C. in a cold bath (2 hours). The solution was re-cooled to −70° C. and (ortho-F—C₆H₄)₂PCI (1.00 g, 3.90 mmol) was slowly added. The solution was stirred for 1 hour at −70° C. and allowed to slowly warm to room temperature forming a white precipitate. The slurry was pumped to dryness and the residue was extracted with toluene and filtered. The filtrate was pumped to dryness and recrystallized from heptane at −70° C. (2×) yielding 1.13 g (58%) of product. At room temperature this material was an oil which contained both the desired ligand (ortho-F—C₈H₄)₂PN(i-Pr)P(ortho-F—C₈H₄)₂ and its isomer (ortho-F—C₆H₄)₂P[═N(i-Pr]P(ortho-F—C₆H₄)₂. A toluene solution of this mixture and 50 mg of (ortho-F—C₆H₄)₂PCI was heated at 65° C. for three hours to convert the isomer to the desired ligand. ¹H NMR (THF-d₈, δ): 7.35 (m, 8H), 7.10 (m, 4H), 6.96 (m, 4H), 3.94 (m, 1H), 1.24 (d, 6H, J=7 Hz). ¹⁹F NMR (THF-d₈, δ): −104.2 (br. s).

In a more preferred procedure the initial steps of the synthesis are conducted in pentane at −5° C. (instead of ether) with 10% more of the (ortho-F—C₆H₄)₂PCI (otherwise as described above). This preferred procedure allows (ortho-F—C₆H₄)₂PN(i-Pr)P(ortho-F—C₆H₄)₂ to be formed in high (essentially quantitative) yield without the final step of heating in toluene.

### Catalyst Preparation

The term catalyst refers to the chromium molecule with the heteroatom ligand bonded in place. The preferred P—N—P ligand does not easily react with some Cr (III) molecules—especially when using the most preferred P—N—P ligands (which ligands contain phenyl groups bonded to the P atoms, further characterized in that at least one of the phenyl groups contains an ortho fluoro substituent).

While not wishing to be bound by theory, it is believed that the reaction between the ligand and the Cr species is facilitated by aluminum alkyl or MAO. It is also believed that the reaction is facilitated by an excess of Al over Cr. Accordingly, it is most preferred to add the Cr/ligand mixture to the MAO (and/or Al alkyl) instead of the reverse addition sequence. In this manner, the initiation of the reaction is believed to be facilitated by the very high Al/Cr ratio that exists when the first part of the Cr/ligand is added to the MAO.

In a similar vein, it is believed that the ligand/Cr ratio provides another kinetic driving force for the reaction—i.e. the reaction is believed to be facilitated by high ligand/Cr ratios. Thus, one way to drive the reaction is to use an excess of ligand. In another, (preferred) reaction, a mixture with a high ligand/Cr ratio is initially employed, followed by lower ligand/Cr ratio mixtures, followed by Cr (in the absence of ligand).

### Part II: Oligomerization Reaction

### General

The aluminoxane used in all experiments was purchased from Albemarle Corporation and reported to contain 10 weight % aluminum. The product was described as a conventional methylaluminoxane that was prepared using TMA as the only source of an aluminum (i.e., it was not a so-called “modified MAO”). The “free TMA” content was reported to be about 10 mole %—i.e. for every 100 moles of aluminum in the product, 90 moles were contained in the aluminoxane oligomer and 10 were present as “free TMA”. For convenience, this product is referred to as “MAO” in the accompanying table and detailed experimental description. (For further certainty: the “Al(MAO)” column includes the aluminum contained in both the aluminoxane oligomer and free TMA. For example, the value of 1,000 micromoles—for inventive run 17—represents 900 micromoles of aluminum in the oligomer and 100 micromoles of free TMA.)

The runs represent four different conditions. Comparative Run 1 (example 1, below) illustrates an oligomerization reaction that was conducted in octene-1 using a conventional chromium concentration of about 40 micromoles and standard MAO activation. Comparative Example 2 (runs 2-9) confirms that the activity can be increased by using cyclohexane solvent at these Cr concentrations.

Comparative Example 3 (runs 10-16) shows that the addition of TEAL can also produce active oligomerizations.

Inventive Example 4, (run 17) shows that very high activity can be achieved in octene when using low Cr concentrations and added TEAL. Note that the activity in Example 4 is higher than that of Example 3—i.e. the activity is higher in the absence of cyclohexane at low Cr concentrations (whereas the opposite was observed at higher Cr concentrations). In addition, the activity of this inventive run is greater than 3×10⁶ grams of product/gram chromium per hour. One advantage of this invention is that it facilitates a bulk oligomerization process—i.e. high activity is achieved in the absence of the cyclohexane solvent.

### EXAMPLES

### Comparative Run 1—Baseline Run in 1-Octene; Standard [Cr]

A 600 mL reactor fitted with a stirrer was purged 3 times with argon while heated at 80° C. The reactor was then cooled to 55° C. (−5° C. below reaction temperature) and a solution of MAO (1.44 g, 10 weight % MAO) in 65 g of 1-octene (containing 5.97 weight % cyclohexane as internal reference) was transferred via a stainless steel cannula to the reactor, followed by 78 g of 1-octene (containing 5.97 weight % cyclohexane). Stirrer was started and set to 1700 rpm. The reactor was then pressurized to 39 bar with ethylene and temperature adjusted to 47° C. Ligand 1 (4.22 mg, 0.0084 mmol) and chromium acetylacetonate (2.88 mg, 0.0082 mmol) were premixed in 14.3 g of 1-octene (containing 5.97 weight % cyclohexane) in a hypovial. The mixture was transferred under ethylene to the pressurized reactor and then the reactor pressure was immediately increased to 45 bar with ethylene. The reaction was allowed to proceed for 20 minutes while maintaining the temperature at 60° C. The reaction was terminated by stopping ethylene flow to the reactor and cooling the contents to 30° C. Stirring was stopped and reactor slowly depressurized to atmospheric pressure. Reactor was then opened and product mixture transferred to a pre-weighed flask containing 1.5 g of isopropanol. The mass of product produced was 85.6 g. A sample of the liquid product was analyzed by GC-FID.

### Example 2

### Baseline Run in Cyclohexane; Runs 2-9

(BSR6Run#1146 (Runs 1173, 1174, 1175, 1176, 1177, 1178 and 1179 follow same procedure as example 2 with varying Cr and Al concentrations)

A 600 mL reactor fitted with a stirrer was purged 3 times with argon while heated at 80° C. The reactor was then cooled to 42° C. (˜5° C. below reaction temperature) and a solution of MAO (1.44 g, 10 weight % MAO) in 65 g of cyclohexane was transferred via a stainless steel cannula to the reactor, followed by 78 g of cyclohexane. Stirrer was started and set to 1700 rpm. The reactor was then pressurized to 35 bar with ethylene and temperature adjusted to 47° C. Ligand 1 (4.43 mg, 0.0089 mmol) and chromium acetylacetonate (3.02 mg, 0.0087 mmol) were premixed in 14.3 g of cyclohexane in a hypovial. The mixture was transferred under ethylene to the pressurized reactor and then the reactor pressure was immediately increased to 40 bar with ethylene. The reaction was allowed to proceed for 15 minutes while maintaining the temperature at 46° C. The reaction was terminated by stopping ethylene flow to the reactor and cooling the contents to 30° C. Stirring was stopped and reactor slowly depressurized to atmospheric pressure. Reactor was then opened and product mixture transferred to a pre-weighed flask containing 1.5 g of isopropanol. The mass of product produced was 100.3 g. A sample of the liquid product was analyzed by GC-FID.

### Example 3

### MAO/TEAL Run in Cyclohexane; Runs 10-16

(BSR6Run#1180 (Runs 1181, 1182, 1183, 1184, 1185, 1186 and 1193 follow same procedure as example 3 with varying TEAL:MAO ratios.)

A 600 mL reactor fitted with a stirrer was purged 3 times with argon while heated at 80° C. The reactor was then cooled to 42° C. (˜5° C. below reaction temperature) and a solution of MAO (0.171 g, 10 weight % MAO) and TEAL (0.0315 g, 0.276 mmol) in 65 g of cyclohexane was transferred via a stainless steel cannula to the reactor, followed by 78 g of cyclohexane. Stirrer was started and set to 1700 rpm. The reactor was then pressurized to 35 bar with ethylene and temperature adjusted to 47° C. Ligand 1 (0.485 mg, 0.001 mmol) and chromium acetylacetonate (0.324 mg, 0.00093 mmol) were premixed in 14.3 g of cyclohexane in a hypovial. The mixture was transferred under ethylene to the pressurized reactor and then the reactor pressure was immediately increased to 40 bar with ethylene. The reaction was allowed to proceed for 45 min. while maintaining the temperature at 47° C. The reaction was terminated by stopping ethylene flow to the reactor and cooling the contents to 30° C. Stirring was stopped and reactor slowly depressurized to atmospheric pressure. Reactor was then opened and product mixture transferred to a pre-weighed flask containing 1.5 g of isopropanol. The mass of product produced was 104.1 g. A sample of the liquid product was analyzed by GC-FID.

### Example 4

### TEAL/MAO Run in 1-Octene

### (BSR6Run#1199)

A 600 mL reactor fitted with a stirrer was purged 3 times with argon while heated at 80° C. The reactor was then cooled to 55° C. (˜5° C. below reaction temperature) and a solution of MAO (0.133 g, 10 weight % MAO) and TEAL (0.0421 g, 0.369 mmol) in 65 g of 1-octene (containing 5.78 weight % cyclohexane as internal reference) was transferred via a stainless steel cannula to the reactor, followed by 78 g of 1-octene (containing 5.78 weight % cyclohexane). Stirrer was started and set to 1700 rpm. The reactor was then pressurized to 39 bar with ethylene and temperature adjusted to 47° C. Ligand 1 (0.484 mg, 0.00097 mmol) and chromium acetylacetonate (0.327 mg, 0.00094 mmol) were premixed in 14.3 g of 1-octene (containing 5.78 weight % cyclohexane) in a hypovial. The mixture was transferred under ethylene to the pressurized reactor and then the reactor pressure was immediately increased to 45 bar with ethylene. The reaction was allowed to proceed for 37 minutes while maintaining the temperature at 60° C. The reaction was terminated by stopping ethylene flow to the reactor and cooling the contents to 30° C. Stirring was stopped and reactor slowly depressurized to atmospheric pressure. Reactor was then opened and product mixture transferred to a pre-weighed flask containing 1.5 g of isopropanol. The mass of product produced was 94.8 g. A sample of the liquid product was analyzed by GC-FID.

TABLE 1

| | | | | | | Activity | PE wt % | | | |
|---|---|---|---|---|---|---|---|---|---|---|
| | Al:Cr | | | | | gProduct/gCr/hr | (based on | | | C10 and |
| | Ratio | Cr | Total Al | Al(MAO) | Al(TEAL) | (based on | isolated | C6 | C8 | higher |
| Runs | (mol:mol) | (microM) | (microM) | (microM) | (microM) | isolated product) | product) | (wt %) | (wt %) | (wt %) |
| 1 | 300 | 41.4 | 12419 | 12419 | 0.0 | 599,011 | 11.4 | 18.0 | 66.8 | 15.2 |
| 2 | 300 | 47.3 | 14187 | 14187 | 0.0 | 891,275 | 2.3 | 16.6 | 70.2 | 13.1 |
| 3 | 312 | 45.1 | 14063 | 14063 | 0.0 | 767,288 | 1.6 | 16.6 | 73.7 | 9.6 |
| 4 | 310 | 15.4 | 4773 | 4773 | 0.0 | 698,142 | 10.0 | 17.3 | 75.1 | 7.6 |
| 5 | 315 | 10.7 | 3361 | 3361 | 0.0 | 555,568 | 7.4 | 18.2 | 74.8 | 7.0 |
| 6 | 314 | 5.0 | 1572 | 1572 | 0.0 | 184,128 | 75.0 | 18.9 | 74.5 | 6.5 |
| 7 | 1240 | 5.0 | 6212 | 6212 | 0.0 | 1,971,279 | 2.1 | 17.0 | 74.6 | 8.3 |
| 8 | 601 | 5.1 | 3093 | 3093 | 0.0 | 957,052 | 11.0 | 18.8 | 74.1 | 7.0 |
| 9 | 902 | 5.1 | 4554 | 4554 | 0.0 | 1,304,500 | 4.8 | 17.6 | 74.2 | 8.1 |
| 10 | 615 | 5.1 | 3123 | 1562 | 1562 | 2,879,330 | 1.5 | 16.6 | 73.0 | 10.3 |
| 11 | 405 | 5.1 | 2069 | 1035 | 1035 | 1,788,554 | 2.1 | 17.8 | 73.2 | 8.9 |
| 12 | 613 | 5.1 | 3107 | 1036 | 2072 | 1,769,502 | 2.0 | 16.5 | 73.9 | 9.5 |
| 13 | 595 | 5.1 | 3021 | 755 | 2266 | 900,604 | 2.3 | 16.5 | 74.7 | 8.7 |
| 14 | 616 | 5.1 | 3125 | 1042 | 2083 | 1,427,882 | 2.2 | 16.4 | 74.1 | 9.4 |
| 15 | 613 | 5.1 | 3114 | 1038 | 2076 | 1,376,785 | 1.4 | 16.7 | 74.2 | 9.0 |
| 16 | 617 | 5.1 | 3132 | 1044 | 2088 | 1,756,361 | 0.9 | 16.6 | 74.5 | 9.8 |
| 17 | 638 | 4.7 | 3001 | 1000 | 2000 | 3,155,057 | 3.4 | 25.9 | 58.8 | 15.2 |

### Continuous Oligomerization

A series of continuous oligomerization experiments was conducted in a one liter reactor. The reactor was equipped with an agitator; and inlet part for feed and an outlet part for oligomer product. The catalyst used was the same as that used for the batch experiments. The activator system consisted of MAO (containing about 20 mole % free TMA, according to product specifications from the supplier) and additional TEAL. The catalyst, MAO and TEAL were added continuously to the reactor (with the MAO and TEAL being “pre-contacted” by way of being co-fed through a common feed line).

Highly active oligomerization reactions were observed over a temperature range of from 50 to 90° C. and at pressures of up to 90 atmospheres, particularly when using comparatively low Cr concentrations and high TEAL ratios (in comparison to the batch reactions of the previous example). Relatively low levels of polymer formation were typically observed. Hydrogen was used in several of the experiments and was observed to further reduce the amount of polymer being produced.

Optimized reaction conditions were observed at Cr concentrations of 0.6 to 3×10⁻⁶ moles per liter (especially 1-2×10⁻⁶); Al/Cr ratio of 1000-1500/1 with the Al being provided by roughly equivalent amounts of MAO and TEAL. The use of 600 moles of MAO (including TMA) and 700 moles of TEAL provided excellent results.

Comparative experiments (using cyclohexane as a solvent) were also observed to be highly active under these conditions.

### INDUSTRIAL APPLICABILITY

This invention enables the “bulk” oligomerization of ethylene (i.e. the oligomerization of ethylene in the presence of the oligomer product) using a catalyst system comprising 1) a very low concentration of a chromium catalyst and 2) a three part activator. The chromium catalyst contains a diphosphine ligand, preferably a so called P—N—P ligand. The activator includes an aluminoxane, trimethyl aluminum, and triethyl aluminum. The process reduces total energy consumption per unit of oligomer produced because it reduces/eliminates the need to separate a process solvent from the oligomer product. The process relies on the use of higher relative levels of triethyl aluminum (and correspondingly lower relative levels of trimethyl aluminum) in comparison to prior art processes. This may provide some cost advantage as triethyl aluminum is generally lower in price than trimethyl aluminum. The linear octene and hexene oligomers that are produced by this process are suitable for use as comonomers for the production of ethylene-alpha olefin copolymers.

1. A process for the oligomerization of ethylene, said process comprising contacting ethylene with
1) an oligomerization catalyst comprising
1.1) a ligand defined by the formula (R¹)(R²)—P¹-bridge-P²(R³)(R⁴) wherein R¹, R², R³ and R⁴ are independently selected from the group consisting of hydrocarbyl and heterohydrocarbyl and the bridge is a divalent moiety that is bonded to both phosphorus atoms; and
1.2) a source of chromium that coordinates to said ligand;
2) a three part activator comprising:
2.1) an aluminoxane;
2.2) trimethyl aluminum; and
2.3) triethyl aluminum;
where said aluminoxane, said trimethyl aluminum and said triethylaluminum are contacted with each other prior to contacting said catalyst and
wherein said process is conducted oligomerization conditions in an oligomerization reactor, with the further proviso that
A) said process is conducted in a liquid that contains more than 50 weight % octene; and
B) said chromium is contained in said process at a concentration of from 0.5 to 8×10⁻⁶ gram moles per litre.

2. The process according to claim 1 wherein said bridge is —N(R⁵)— wherein R⁵ is selected from the group consisting of hydrogen, alkyl, substituted alkyl, aryl, substituted aryl, aryloxy, substituted aryloxy, halogen, alkoxycarbonyl, carbonyloxy, alkoxy, aminocarbonyl, carbonylamino, dialkylamino, silyl groups or derivatives thereof and an aryl group substituted with any of these substituents.

3. The process according to claim 1 wherein said aluminoxane is methylaluminoxane.

4. The process according to claim 1 wherein said process is conducted as a bulk oligomerization process.

5. The process according to claim 1 wherein hydrogen is added.

6. The process according to claim 1 wherein said oligomerization conditions comprise a temperature of from 10 to 100° C. and a pressure of from 5 to 100 atmospheres.

7. The process according to claim 2 where R⁵ is isopropyl and R¹ and R³ are ortho-fluoro phenyl.

8. The process according to claim 7 wherein R2 and R4 are ortho-fluoro phenyl.

9. The process according to claim 1, further characterized in that the oligomerization rate is greater than 3 million grams of ethylene consumed per hour per gram of chromium.
